SEQUENCE LISTING (91 sequences)

SEQ ID NO: 1 - protein, 98 aa, Homo sapiens
  Seq ID No. 1 VH1-02
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 2 - DNA, 294 bp, Homo sapiens
  source 1..294: /organism=“Homo sapiens” /note=“Seq ID No. 2 VH1-02 ” /mol_type=“unassigned DNA”
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgaga

SEQ ID NO: 3 - protein, 121 aa, Homo sapiens
  Seq ID No. 3 VH 7D7
QVQLVQSGAEVKKPGASVKVSCKASGYTFTGYNMHWVRQAPGQGLEWMGWINPNSGGTNYAQKFQGRVTMTRDTSISTAYMELSRLRSDDTAVYYCAREGSSTVTREMDVWGQGTTVTVSS

SEQ ID NO: 4 - DNA, 363 bp, Homo sapiens
  source 1..363: /organism=“Homo sapiens” /note=“Seq ID No. 4 VH 7D7 ” /mol_type=“unassigned DNA”
caggtgcagctggtgcagtctggggctgaggtgaagaagcctggggcctcagtgaaggtctcctgcaaggcttctggatacaccttcacaggctacaatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagaggggtcatctacagtaaccagggagatggacgtctggggccaagggaccacggtcaccgtctcttca

SEQ ID NO: 5 - protein, 128 aa, Homo sapiens
  Seq ID No. 5 VH 6B2
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARAYCGGDCPQDYYDYYGMDVWGQGTTVTVSS

SEQ ID NO: 6 - DNA, 384 bp, Homo sapiens
  source 1..384: /organism=“Homo sapiens” /note=“Seq ID No. 6 VH 6B2 ” /mol_type=“unassigned DNA”
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagcatattgtggtggtgactgcccccaggattactatgactactacggtatggacgtctggggccaagggaccacggtcaccgtctcttca

SEQ ID NO: 7 - protein, 124 aa, Homo sapiens
  Seq ID No. 7 VH 129D2
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCASRPLGYCGDGSCPFDYWGQGTLVTVSS

SEQ ID NO: 8 - DNA, 372 bp, Homo sapiens
  source 1..372: /organism=“Homo sapiens” /note=“Seq ID No. 8 VH 129D2 ” /mol_type=“unassigned DNA”
caggtccagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagccgacccttggggtattgtggtgatggcagctgcccctttgactactggggccagggaaccctggtcactgtctcttca

SEQ ID NO: 9 - DNA, 21 bp, Artificial Sequence
  source 1..21: /organism=“Artificial Sequence” /note=“PCR Primer” /mol_type=“unassigned DNA”
gtccgggagatcatgagagtg

SEQ ID NO: 10 - DNA, 42 bp, Artificial Sequence
  source 1..42: /organism=“Artificial Sequence” /note=“PCR Primer” /mol_type=“unassigned DNA”
ggaacagaccaccatggcccaggtbcagctggtgcagtctgg

SEQ ID NO: 11 - DNA, 42 bp, Artificial Sequence
  source 1..42: /organism=“Artificial Sequence” /note=“PCR Primer” /mol_type=“unassigned DNA”
ggaacagaccaccatggcccagatcaccttgaaggagtctgg

SEQ ID NO: 12 - DNA, 42 bp, Artificial Sequence
  source 1..42: /organism=“Artificial Sequence” /note=“PCR Primer” /mol_type=“unassigned DNA”
ggaacagaccaccatggcccaggtgcagctgcaggagtcggg

SEQ ID NO: 13 - DNA, 42 bp, Artificial Sequence
  source 1..42: /organism=“Artificial Sequence” /note=“PCR Primer” /mol_type=“unassigned DNA”
ggaacagaccaccatggcccaggtacagctgcagcagtcagg

SEQ ID NO: 14 - DNA, 34 bp, Artificial Sequence
  source 1..34: /organism=“Artificial Sequence” /note=“PCR Primer” /mol_type=“unassigned DNA”
  misc_feature 22: /note=“r = g or a”
  misc_feature 28: /note=“r = g or a”
gctaccgccaccctcgagtgargagacrgtgacc

SEQ ID NO: 15 - DNA, 30 bp, Artificial Sequence
  source 1..30: /organism=“Artificial Sequence” /note=“PCR Primer” /mol_type=“unassigned DNA”
gtccatggccatcgccggctgggccgcgag

SEQ ID NO: 16 - DNA, 49 bp, Artificial Sequence
  source 1..49: /organism=“Artificial Sequence” /note=“PCR Primer” /mol_type=“unassigned DNA”
tagcagcctcgagggtggcggtagccatcaccaccatcaccacgggagc

SEQ ID NO: 17 - DNA, 50 bp, Artificial Sequence
  source 1..50: /organism=“Artificial Sequence” /note=“PCR Primer” /mol_type=“unassigned DNA”
  misc_feature 25: /note=“b = g or c or t/u”
ggaacagaccaccatggcccaggtbcagctggtgcagtctggggctgagg

SEQ ID NO: 18 - DNA, 23 bp, Artificial Sequence
  source 1..23: /organism=“Artificial Sequence” /note=“PCR Primer” /mol_type=“unassigned DNA”
gacacggccgtgtattactgtgc

SEQ ID NO: 19 - DNA, 23 bp, Artificial Sequence
  source 1..23: /organism=“Artificial Sequence” /note=“PCR Primer” /mol_type=“unassigned DNA”
gcacagtaatacacggccgtgtc

SEQ ID NO: 20 - protein, 15 aa, Homo sapiens
  CDR3 sequence
AARPGTLYYFHEIDV

SEQ ID NO: 21 - protein, 15 aa, Homo sapiens
  CDR3 sequence
APGWVDGRYFHQLEN

SEQ ID NO: 22 - protein, 20 aa, Homo sapiens
  CDR3 sequence
APIGAVPTAFNIFYYHYMDV

SEQ ID NO: 23 - protein, 21 aa, Homo sapiens
  CDR3 sequence
DPGGYYNPRQETFYYYHHMDV

SEQ ID NO: 24 - protein, 16 aa, Homo sapiens
  CDR3 sequence
GAFDYGDPPPFHYMDV

SEQ ID NO: 25 - protein, 18 aa, Homo sapiens
  CDR3 sequence
GGLDDSGYPRLLPHYFDY

SEQ ID NO: 26 - protein, 14 aa, Homo sapiens
  CDR3 sequence
GTLSSGYYYHYFDY

SEQ ID NO: 27 - protein, 14 aa, Homo sapiens
  CDR3 sequence
LPKDYNAYQYGLDV

SEQ ID NO: 28 - protein, 14 aa, Homo sapiens
  CDR3 sequence
LPKDYNSYQYGLDV

SEQ ID NO: 29 - protein, 14 aa, Homo sapiens
  CDR3 sequence
LPKYYNSYQYGLDV

SEQ ID NO: 30 - protein, 14 aa, Homo sapiens
  CDR3 sequence
LPRTYYDNTGAFDQ

SEQ ID NO: 31 - protein, 17 aa, Homo sapiens
  CDR3 sequence
MGSNWPHEGYYYHYMDV

SEQ ID NO: 32 - protein, 13 aa, Homo sapiens
  CDR3 sequence
QEGLVDSYYGMDV

SEQ ID NO: 33 - protein, 15 aa, Homo sapiens
  CDR3 sequence
RPLGYCGDGSCPFDY

SEQ ID NO: 34 - protein, 10 aa, Homo sapiens
  CDR3 sequence
SIAATRPNYA

SEQ ID NO: 35 - protein, 16 aa, Homo sapiens
  CDR3 sequence
TSGSGNYLPYHYAMDV

SEQ ID NO: 36 - protein, 5 aa, Homo sapiens
  CDR3 sequence
AFGNY

SEQ ID NO: 37 - protein, 19 aa, Homo sapiens
  CDR3 sequence
AKTGLHLGELSQTRVYFDY

SEQ ID NO: 38 - protein, 12 aa, Homo sapiens
  CDR3 sequence
ARGYTGRVYFDN

SEQ ID NO: 39 - protein, 14 aa, Homo sapiens
  CDR3 sequence
ARSERGYGRHYFDH

SEQ ID NO: 40 - protein, 18 aa, Homo sapiens
  CDR3 sequence
ASLGTGFWSGYSRHYFDN

SEQ ID NO: 41 - protein, 12 aa, Homo sapiens
  CDR3 sequence
AVGEGPYAHADH

SEQ ID NO: 42 - protein, 13 aa, Homo sapiens
  CDR3 sequence
DDGTTGSRRSLEI

SEQ ID NO: 43 - protein, 17 aa, Homo sapiens
  CDR3 sequence
DITGDLGRSGYRSYFDY

SEQ ID NO: 44 - protein, 18 aa, Homo sapiens
  CDR3 sequence
DKGGAYCSGGNCYYPFDY

SEQ ID NO: 45 - protein, 21 aa, Homo sapiens
  CDR3 sequence
DMSPVIAPLLGKTRYYYVMDV

SEQ ID NO: 46 - protein, 17 aa, Homo sapiens
  CDR3 sequence
DRDFWSGFYHHRSYFDY

SEQ ID NO: 47 - protein, 16 aa, Homo sapiens
  CDR3 sequence
DRGGIVGTKVRRHLDY

SEQ ID NO: 48 - protein, 14 aa, Homo sapiens
  CDR3 sequence
DTRPRTSSRRYFDY

SEQ ID NO: 49 - protein, 13 aa, Homo sapiens
  CDR3 sequence
EALVSSTRHYFDS

SEQ ID NO: 50 - protein, 13 aa, Homo sapiens
  CDR3 sequence
EGYSSGPKYFFDY

SEQ ID NO: 51 - protein, 13 aa, Homo sapiens
  CDR3 sequence
EIFFTSGRRFLDP

SEQ ID NO: 52 - protein, 14 aa, Homo sapiens
  CDR3 sequence
EIGTSGSYRRYFDS

SEQ ID NO: 53 - protein, 14 aa, Homo sapiens
  CDR3 sequence
ERDSNYANKHYFDY

SEQ ID NO: 54 - protein, 15 aa, Homo sapiens
  CDR3 sequence
ERGGPFGAARHYFDY

SEQ ID NO: 55 - protein, 13 aa, Homo sapiens
  CDR3 sequence
ERSRTPFRRYFDF

SEQ ID NO: 56 - protein, 15 aa, Homo sapiens
  CDR3 sequence
ERYFDSSTYRRYFDS

SEQ ID NO: 57 - protein, 13 aa, Homo sapiens
  CDR3 sequence
ESTASPSRRYLDY

SEQ ID NO: 58 - protein, 15 aa, Homo sapiens
  CDR3 sequence
GAQIASAATRHYFDY

SEQ ID NO: 59 - protein, 17 aa, Homo sapiens
  CDR3 sequence
GEYDYVRGTYTYFGLDI

SEQ ID NO: 60 - protein, 5 aa, Homo sapiens
  CDR3 sequence
GFGNV

SEQ ID NO: 61 - protein, 11 aa, Homo sapiens
  CDR3 sequence
GGASARYYFDY

SEQ ID NO: 62 - protein, 10 aa, Homo sapiens
  CDR3 sequence
GGQLRVALDN

SEQ ID NO: 63 - protein, 8 aa, Homo sapiens
  CDR3 sequence
GKHGYATH

SEQ ID NO: 64 - protein, 12 aa, Homo sapiens
  CDR3 sequence
GKWDGTRYLLVY

SEQ ID NO: 65 - protein, 16 aa, Homo sapiens
  CDR3 sequence
GMKGYYGSSRRYYFDY

SEQ ID NO: 66 - protein, 16 aa, Homo sapiens
  CDR3 sequence
GPPYCSDIGCYTAGDY

SEQ ID NO: 67 - protein, 14 aa, Homo sapiens
  CDR3 sequence
GQIRNWNYGGTLRD

SEQ ID NO: 68 - protein, 13 aa, Homo sapiens
  CDR3 sequence
GRRSGSGRVYFDN

SEQ ID NO: 69 - protein, 15 aa, Homo sapiens
  CDR3 sequence
GTGYDSSGYYQRADK

SEQ ID NO: 70 - protein, 13 aa, Homo sapiens
  CDR3 sequence
GVAARPGRAYLDS

SEQ ID NO: 71 - protein, 15 aa, Homo sapiens
  CDR3 sequence
GVESVRRRRNGYLDP

SEQ ID NO: 72 - protein, 12 aa, Homo sapiens
  CDR3 sequence
HRGYSYTSPFDY

SEQ ID NO: 73 - protein, 13 aa, Homo sapiens
  CDR3 sequence
LHYSNSWTGPSDY

SEQ ID NO: 74 - protein, 22 aa, Homo sapiens
  CDR3 sequence
MTSKKYILTGHYKGSNYYAMDV

SEQ ID NO: 75 - protein, 4 aa, Homo sapiens
  CDR3 sequence
PGGP

SEQ ID NO: 76 - protein, 14 aa, Homo sapiens
  CDR3 sequence
QLTKVAAGRRYFDS

SEQ ID NO: 77 - protein, 17 aa, Homo sapiens
  CDR3 sequence
RAAHHLTGQPARYPLDV

SEQ ID NO: 78 - protein, 4 aa, Homo sapiens
  CDR3 sequence
RHVA

SEQ ID NO: 79 - protein, 14 aa, Homo sapiens
  CDR3 sequence
SHYDNVSGYFTSDK

SEQ ID NO: 80 - protein, 13 aa, Homo sapiens
  CDR3 sequence
SPPAVVKKYYFDY

SEQ ID NO: 81 - protein, 12 aa, Homo sapiens
  CDR3 sequence
SRTVGRSYPFDY

SEQ ID NO: 82 - protein, 16 aa, Homo sapiens
  CDR3 sequence
TKEQWGLVGSRVHFDY

SEQ ID NO: 83 - protein, 13 aa, Homo sapiens
  CDR3 sequence
VASERVTYGAFDI

SEQ ID NO: 84 - protein, 13 aa, Homo sapiens
  CDR3 sequence
VAVEARPYGGLDI

SEQ ID NO: 85 - protein, 18 aa, Homo sapiens
  CDR3 sequence
VGRKYFYERSGYNDVFDF

SEQ ID NO: 86 - protein, 10 aa, Homo sapiens
  CDR3 sequence
VKGGRGRMDV

SEQ ID NO: 87 - protein, 12 aa, Homo sapiens
  CDR3 sequence
VLGTGTTGPAVY

SEQ ID NO: 88 - protein, 18 aa, Homo sapiens
  CDR3 sequence
VMGVPVTSTNRPRHIFDL

SEQ ID NO: 89 - protein, 13 aa, Homo sapiens
  CDR3 sequence
VRDTFGYRHYFDS

SEQ ID NO: 90 - protein, 12 aa, Homo sapiens
  CDR3 sequence
VRGSGRWGAFDY

SEQ ID NO: 91 - protein, 12 aa, Homo sapiens
  CDR3 sequence
VSGRGPFGHADY

(19) United States
(12) Patent Application Publication
(10) Pub. No.: US 2017/0306319 A1
(43) Pub. Date: Oct. 26, 2017

(54) VH SCAFFOLD

(71) Applicant: Crescendo Biologics Limited, Cambridge, Cambridgeshire (GB)

(72) Inventor: Bryan Edwards, Cambridge (GB)

(73) Assignee: Crescendo Biologics Limited, Cambridge, Cambridgeshire (GB)

(21) Appl. No.: 15/520,617

(22) PCT Filed: Oct. 22, 2014
(86) PCT No.: PCT/GB2014/053143

§ 371 Date: Apr. 20, 2017

(51) Int. Cl. C12N 15/10; C07K 16/24; C07K 16/00

(52) U.S. Cl. CPC C12N 15/1041; C07K 16/005; C07K 16/24; C07K 2317/567; C07K 16/00; C07K 2317/21; C07K 2317/569; C07K 2317/76; C07K 2317/565

(57) ABSTRACT

The invention provides human VH scaffold sequences, libraries derived therefrom and methods of producing. The scaffolds have high expression, solubility and are functional.

### FIELD OF THE INVENTION

The invention relates to a novel human VH domain scaffold, libraries derived from the scaffold, methods of construction and pharmaceutical compositions comprising the VH domain scaffold.

### BACKGROUND TO THE INVENTION

Most natural conventional antibodies or immunoglobulins (Ig's) are tetrameric molecules made up of paired heterodimers (each comprising one heavy and one light chain) stabilised and cross-linked by inter-chain and intra-chain disulphide bonds. The light chains may be of either the kappa or lambda isotype. Each of the heavy and light chains fold into domains, each light chain having an N-terminal variable (VL) and a C-terminal constant domain (CL) which may be either Cκ or Cλ. Each heavy chain comprises an N-terminal variable (VH) domain followed by a first constant domain (CH1) a hinge domain and two or three further constant domains (CH2, CH3 and optionally CH4). Association of the VH domain on each heavy chain with the VL domain on its partner light chain results in the formation of two antigen binding regions (Fv). Interaction between the CH1 domain and the CL domain is known to facilitate functional association between the heavy and light chains. Each Fv region comprises an antigen binging site formed by six hypervariable polypeptide loops or complementarity determining regions (CDRs), three derived from the VH domain (H1, H2 and H3) and three from the VL domain (L1, L2 and L3). The CDRs interact directly with antigen. The scaffold sequences in the Fv which support the CDRs are known as framework regions (FRs).

The VH domain is encoded by gene segments located in the heavy chain locus. Similarly the VL domain is encoded by gene segments located in one of the two light chain loci. During normal B-cell development, one of a multitude of VH gene segments is rearranged with one of a number of D-gene segments and one of a number of J-gene segments, the final VDJ arrangement encoding a complete VH region. The majority of the VH region (including CDRs 1 and 2) is encoded by the VH gene segment. The D-J combination encodes the rest of the VH region (in particular CDR3). Combinatorial choice of exactly which V-, D- and J-gene-segments are used, imprecision of the D-J join and somatic hypermutation all result in significant sequence diversity focused in the heavy chain CDRs. In particular, the heavy chain CDR3 acquires greatest sequence diversity and therefore generally contributes the most to antibody specificity. The light chains undergo a similar process, recombining one light chain V-gene segment with one light chain J-gene segment to form the VL sequence. Combinatorial sequence diversity is once again focused in the VL CDRs. The constant regions of both the heavy and light chains are relatively invariant.

In conventional antibodies, generally, both the VH and the VL are required for antigen binding. However, camelids (camels, dromedaries and llamas) and certain sharks are known to naturally produce a class of functional antibodies devoid of light chains (Hamers et al 1993). Such heavy-chain only antibodies are distinct from conventional antibodies in that they are homodimers of a heavy chain comprised of a VH and a number of CH domains but importantly they lack a CH1 domain. Camelids, are capable of producing both conventional and heavy-chain only antibodies in response to antigen challenge (indeed they often produce both classes of antibody in a single response to antigen). When raising heavy-chain only antibodies, rather than the standard VH domain, camelids use a special class of heavy chain variable region known as VHH (De Genst et al Dev. Comp. Immunol. 30: 187-198).

However, despite many attractive biophysical characteristics, camelid VHH domains do not have a human amino acid sequence and therefore have the potential to initiate an anti-drug immune response when administered to humans. In view of this, VHH domains are not suitable as effective therapeutic products and significant efforts have been made to overcome the problem by ‘humanising’ the camelid sequence. Importantly, it is frequently the case that in order to avoid loss of binding affinity, specificity and functionality it is necessary to retain many original camelid residues. As such, the product destined for therapeutic use in humans will always retain non-human residues.

Consequently there has been a great deal of interest in producing human VH (or VL) domains as therapeutic candidates. It is well known that VH domains derived from conventional antibodies require a companion VL domain and in the absence of the partner domain are difficult to express, often insoluble and suffer loss of binding affinity and specificity to target antigen.

Isolated human VH (or VL) domains require significant engineering in order to enhance solubility and stability. This problem has been approached in a number of ways, for example by ‘camelising’ the human sequence (Davies and Reichmann 1996 Protein Eng 9(6):531-537; Reichmann L and Muyldermans S 1999 J Immunol Methods 231:25-38). Indeed, the requirement for significant engineering to enhance solubility and stability of isolated human VH (or VL) domains means that deriving drug quality therapeutic candidates has been extremely challenging.

Libraries of the prior art have attempted to overcome these limitations, for example US 2011/0052565 describes libraries of non-aggregating human VH domains comprising at least one di-sulphide cysteine in at least one CDR and having an acidic isoelectric point. Non-aggregating VH domains are selected using a heat denaturation and refolding step since selection based solely on binding was not efficient in yielding functional binders. EP1025218 describes a naïve library of human VH domains, all members having a H1 hypervariable loop canonical structure encoded by VH gene segment DP-47, wherein loop is diversified by changing aa at positions H31, H33 and H35. Each time the VH libraries of EP1025218 are used for selecting on target antigen, they are first screened in accordance with the ability to bind to superantigen protein A, a generic ligand which essentially depletes the library of non-functional or poorly folded members. Subsequent to protein A screening, the depleted antibody repertoire is selected against the target antigen, and further rounds of enrichment for binding to target antigen are performed.

Despite the use of a known functional VH3 gene (DP-47) as the basis for a library, the requirement to remove non-functional members was necessary suggesting that the initial repertoire contained a significant number of defective clones.

Thus, the VH libraries of the prior art are limited by their ability to yield soluble functional clones without additional steps such as protein A selection, the combination of heat denaturation with refolding or significant prior engineering for enhanced solubility and stability. In view of these limitations there is a need to provide further VH domain libraries comprising high numbers of soluble, functional clones which may be selected in a direct and efficient manner.

Conventional antibodies are now well established as highly effective therapeutic agents with sales of $54bn in 2012 expected to continue to grow significantly in the coming years. However, there is increasing demand for exploiting the benefits of alternative formats and smaller fragments in order to derive the next generation of antibody-based therapeutic candidates and in light of this and in view of the above-mentioned problems, there is a need to provide further human VH scaffolds, human VH libraries based on the scaffolds and methods thereof which enable the isolation of soluble, stable, high affinity antibodies with low immunogenicity. The provision of further scaffolds and libraries thereof increases the diversity of potential antibodies that may be obtained against a particular target antigen and therefore increases the probability of isolating a VH domain with the desired affinity and specificity. The scaffold of the present invention provides a valuable contribution to the art and further advances the repertoire of soluble human VH domains available to be screened and progressed for clinical development.

### SUMMARY OF THE INVENTION

According to a first aspect of the invention there is provided a human VH soluble scaffold derived from human germline gene VH1-02 capable of producing a VH domain expression library comprising at least 70% soluble clones. The clones are highly expressed, functional and non-aggregating. The clones may be further characterised by the presence of a single, monomer peak when purified by size exclusion chromatography. The scaffold provides a new soluble framework for the generation of a diverse VH domain expression library.

In one embodiment of the invention there is provided a human VH scaffold or fragments thereof according to Seq ID No. 1 and Seq ID No. 2.

According to a further aspect of the invention there is provided a method for identifying a VH scaffold of the first aspect comprising the steps of:
a) Obtaining a transgenic mouse capable of expressing heavy chain only antibodies (HcAbs) comprising human VH domains,
b) Obtaining a VH domain expression library from the mouse of step a) and expressing in E.coli,
c) Detecting soluble VH domains expressed in b) and
d) Determining the sequence of soluble VH domains to obtain a VH scaffold sequence.

According to a further aspect of the invention there is provided a human VH domain expression library derived from the scaffold of the invention. The library comprises a population of VH clones which are soluble, highly expressed, functional and non-aggregating. The libraries are useful in providing for direct and efficient isolation of VH domain antibodies.

In one embodiment there is provided a human VH domain expression library derived from the scaffold according to Seq ID No. 1 and Seq ID No. 2.

According to a further aspect of the invention there is provided a method of constructing a VH domain expression library comprising the steps of;
a) Assembling the scaffold according to the previous aspects to comprise CDR3 regions
b) Obtaining a VH domain repertoire
c) Expressing the VH domain repertoire and selecting for functional VH domains against target antigen.

In a further aspect of the invention there is provided an isolated human VH domain or fragment thereof comprising a scaffold as defined in the previous aspects.

According to a further aspect of the invention there is provided a pharmaceutical composition comprising a therapeutically effective amount of a VH antibody derived from the VH library of the invention, and a pharmaceutically acceptable excipient.

### BRIEF DESCRIPTION OF THE FIGURES

FIG. 1 shows RT-PCR amplification of full-length human VH domains from cDNA isolated from HC transgenic mice. PCR amplification products were observed at the expected size (approx 400 bp, arrowed).

FIG. 2 shows a schematic diagram of phagemid vector pUCG3.

FIG. 3 shows PCR amplification of pUCG3 vector. A PCR product was observed at the expected size (approx 4600 bp, arrowed).

FIG. 4 shows solubility of VH clones from HC transgenic mice. Soluble VH were observed for each of the germline families tested.

FIG. 5 shows CDR3 sequence diversity of soluble VH isolated from each of the germline families tested. CDR3 diversity is highest in the VH1 family.

FIG. 6 shows SEC traces of VH 7D7 and 6B2.

FIG. 7 shows PCR amplification of human CDR3 domains from cDNA. CDR3 amplification products were observed at the expected size (approx 50 to 100 bp, arrowed).

FIG. 8 shows PCR amplification of the VH1-02 scaffold. VH products were observed at the expected size (approx 300 bp, arrowed).

FIG. 9 shows assembly and pull-through PCR amplification of VH1-02 scaffold plus human CDR3 domains. Full length VH products were observed at the expected size (approx 400 bp, arrowed).

FIG. 10 shows solubility of VH clones from the VH1-02 library. Greater than 70% of VH were expressed with an OD450 nm>0.2.

FIG. 11 shows VH yields from small scale expression studies following purification by affinity chromatography.

FIG. 12 shows anti-TNF-α VH (129D2) inhibits binding of TNF-α to TNFR1 in a competition binding assay. C170 =anti-TNF-α reference dAb.

The invention is described further in the following non-limiting examples.

### DETAILED DESCRIPTION OF THE INVENTION

The inventors have provided a new VH scaffold that is surprisingly soluble and forms the basis for the construction of a diverse library of VH domains which retain the advantageous features of the scaffold, and are soluble, correctly folded, stable and functional.

Scaffolds

According to a first aspect of the invention there is provided a human VH soluble scaffold derived from human germline gene VH1-02 capable of producing a VH domain expression library comprising at least 70% soluble clones.

The presence of soluble clones may be measured by analysis of bacterial periplasmic extracts using techniques known in the art, for example immunoblotting or ELISA. With the appropriate leader sequences present, soluble VH expressed in E.coli are transported to the bacterial periplasmic space. Here they can be extracted and coated directly onto solid supports for detection by ELISA. When using ELISA, the absorbance at 450nm is directly proportional to the amount of VH coated, and therefore gives an indication of VH expression and solubility. The inventors have found that the proportion of clones derived from the libraries of the invention which are defined as soluble according to a reading of between 0.2 and 3 OD at 450 nm in ELISA is at least 70%.

Solubility is known to the skilled person as the maximum amount of solute dissolved in a solvent at equilibrium and may also be referred to herein as the ability of a VH domain to dissolve in an appropriate buffer such as phosphate buffered saline (PBS), Tris buffers, HEPES buffers, carbonate buffers or water and to bind antigen.

VH domains are monomeric and in the absence of a VL partner are characteristically “sticky” tending to form aggregates in solution and binding non-specifically to antigen caused by the exposure of hydrophobic amino acid residues that would normally interact with the light chain. This problem is recognised in the prior art and can result in a significant decrease in the quality and diversity of a library. The VHs of the invention are monomeric in form and do not form aggregates in solution. This is due to the properties of the scaffold sequence which in effect acts as a template, transferring its inherent properties such as high solubility, low propensity to aggregate, stability and functionality to the VH domain antibodies produced from it. The presence of a stable, soluble VH domain in monomeric form may be confirmed by the presence of a single correct peak following size exclusion chromatography (SEC).

The VH scaffold of the invention has been found to result in the isolation of a higher proportion of soluble and correctly folded VH domains from a VH library based on the scaffold as defined herein. The scaffold of the invention is capable of producing a VH domain expression library comprising at least 70% soluble clones which are non-aggregating as defined according to the presence of a single correct monomer peak following size exclusion chromatography (SEC), and are stable and functional as defined by the ability to bind antigen.

The scaffold of the invention provides a new soluble framework for the generation of a diverse VH domain library which does not require additional modifications such as protein A deselection in order to reduce background levels due to significant numbers of non-functional clones.

The term “VH” or “VH domain” as used herein refers to an antibody heavy chain variable domain. This includes human VH domains and VH domains that have been altered, for example by mutagenesis and those which occur naturally.

In one embodiment of the invention there is provided a human VH scaffold or fragment thereof according to Seq ID No. 1, and Seq ID No. 2.

The scaffold is derived from the human VH germline gene VH1-02 (identified in VBASE2 at http://www.vbase2.org/vgene.php?id=humIGHV165; Retter I et al Nucl. Adds Res. (2005) 33 (suppl 1): D671-D674) and is referred to herein as VH1-02.

Scaffold VH1-02 was isolated from a HC transgenic mouse which lacks endogenous murine immunoglobulin loci and comprises a YAC encoding human VH genes, human D genes, human J genes a C-region (lacking the CH1 domain) and known key regulatory elements.

The scaffold of the invention is a soluble scaffold. The scaffold of the invention is suitable for the generation of a diverse VH domain library.

The scaffold of the invention has been found to result in the isolation of a higher proportion of soluble and correctly folded VH domains from a VH library based on the scaffold as defined herein.

The scaffold as defined herein may be referred to as comprising CDR regions 1 and 2, (CDR1 and CDR2). The scaffold may be further modified to comprise CDR3 regions, thus forming a diverse library of VH domains comprising CDR1, CDR2, CDR3 and framework regions (FR1, FR2, FR3 and FR4). The framework regions are known as those regions that represent the structural element of the FV region, outside of the CDR regions.

The framework regions of the scaffold may comprise one or more mutations. The mutations may be in any region of the framework region sequence.

The CDR1 and CDR2 regions of the scaffold may be mutated to improve the characteristics of the VH domain, for example improved affinity, solubility, expression or reduced aggregation. Further diversity may be introduced by general molecular biology techniques known to those skilled in the art including site directed mutagenesis, random mutagenesis, error-prone PCR, insertions and deletions (Ausubel et al, Current Protocols in Molecular Biology, John Wley & Sons, New York 2000).

The invention comprises VH scaffold sequences having at least 80%, 90%, 95%, 98% or 99% amino acid sequence identity with the sequences according to SEQ ID No 1. Percent (%) sequence identity can be determined by methods known in the art. For example mathematical algorithms may be employed to compare amino acid sequence similarity between aligned sequences (Karlin & Altschul, Proc. Natl. Acad. Sci. USA 1990; 87: 2264-2268). Various other programs and software packages may be used including the ALIGN program and the FASTA algorithm (Pearson & Lipman, Proc. Natl. Acad. Sci. USA 1988; 85: 2444-2448). The BLAST program provided by the National Center for Biotechnology Information is also widely used and suitable for the purposes of the present invention.

The scaffold of the invention comprises CDR1 and CDR2 sequences having at least 80%, 90%, 95%, 98% or 99% amino acid sequence identity with the CDR1 and CDR2 sequences according to SEQ ID No 1. Alternatively the scaffold of the invention comprises one of CDR1 or CDR2 sequences having at least 80%, 90%, 95%, 98% or 99% amino acid sequence identity with the CDR1 and CDR2 sequences according to SEQ ID No 1.

The invention also relates to nucleic acid sequences encoding the VH scaffold having at least 80%, 90%, 95%, 98% or 99% sequence identity with the sequences according to SEQ ID No 2.

The scaffold of the invention may comprise one or more CDR1 and CDR2 sequences which are grafted in to replace one or both of the existing CDR regions and may be derived from non-human sources, for example camel or mouse. For example the VH domain may comprise a human framework region and a camelid CDR1 and/or CDR2 region. Alternatively the scaffold may comprise humanised CDR1 and/or CDR2 sequences derived from non-human species such as camel or mouse.

According to a further aspect of the invention there is provided a method for identifying a VH scaffold of the first aspect comprising the steps of:
a) Obtaining a transgenic mouse capable of expressing heavy chain only antibodies (HcAbs) comprising human VH domains,
b) Obtaining a VH domain expression library from the mouse of step a) and expressing in E.coli,
c) Detecting soluble VH domains expressed in b) and
d) Determining the sequence of soluble VH domains to obtain a VH scaffold sequence.

The transgenic mouse (also referred to herein as HC transgenic mouse) is devoid of functional endogenous murine immunoglobulin loci (heavy chain, lambda light chain and kappa light chain). A HC transgenic mouse lacks the ability to produce endogenous murine immunoglobulins and will instead express heavy chain only antibodies comprising human VH domains, devoid of a light chain. For example, the mouse may express heavy chain only antibodies, comprising a human VH domain and an Fc domain derived from a non-human mammal. In a further example the mouse may express heavy chain only antibodies comprising a human VH domain and a human Fc domain. Alternatively the mouse may express heavy chain only antibodies comprising a human VH domain and a murine Fc domain. Heavy chain only antibodies may be obtained from HC transgenic mice expressing human VH and human Fc or human VH and murine Fc domains. Only B cells expressing heavy chain-only antibodies will be expanded in these mice.

The generation of HC transgenic mice is undertaken by functionally silencing murine immunoglobulin loci. Specifically, methods used to silence the mouse heavy chain locus (WO2004/076618 & Ren, L., et al., Genomics 84 (2004), 686-695), the mouse lambda locus (WO03000737 & Zou, X., et al., EJI, 1995, 25, 2154-2162 and the kappa locus (Zou, X., et al., JI 2003 170, 1354-1361) have been described previously. Briefly, large scale deletions of the mouse heavy chain constant region and the mouse lambda chain locus result in silencing of these two immunoglobulin chains. The kappa light chain is silenced via a targeted insertion of a neomycin resistant cassette.

Mice with dual silencing of the endogenous light chains (kappa and lambda) are created by conventional breeding (Zou, X., et al., JI 2003 170, 1354-1361). These light chain-KO mice are further bred with heavy chain KO mice to give triple heterozygous animals for breeding to derive a ‘triple knockout’ (TKO) line.

Yeast artificial chromosomes (YACs) are vectors that can be employed for the cloning of very large DNA inserts in yeast. As well as comprising all three cis-acting structural elements essential for behaving like natural yeast chromosomes (an autonomously replicating sequence (ARS), a centromere (CEN) and two telomeres (TEL)), their capacity to accept large DNA inserts enables them to reach the minimum size (150 kb) required for chromosome-like stability and for fidelity of transmission in yeast cells. The construction and use of YACs is well known in the art (e.g. Bruschi, C. V. and Gjuracic, K. Yeast Artificial Chromosomes, ENCYCLOPEDIA OF LIFE SCIENCES 2002 Macmillan Publishers Ltd, Nature Publishing Group/www.els.net). The transgene present in a HC transgenic mouse is an integrated YAC.

Purified YAC DNA comprising human VH genes, human D genes, human J genes, at least one C-region lacking the CH1 domain and key regulatory elements (known in the art) such as enhancer and switch elementscan be assembled and prepared by methods known in the art (e.g. A. Fernandez, D. Munoz & L. Montoliu in “Generation of Transgenic Animals by Use of YACs” pp 137-158 in “Advanced Protocols for Animal Transgenesis. An ISTT Manual.Ed. Shirley Pease & Thomas L. Saunders. Springer Protocols 2011).

The YAC may have a chimeric heavy chain locus comprising sequences of human and murine origin. The YAC may further comprise a heavy chain constant region which does not encode a CH1 domain and at least 10 functional human heavy chain V genes wherein the at least 10 functional human heavy chain V genes are substantially in their natural configuration. For example the YAC may comprise;
a) at least 10 functional human heavy chain V genes wherein at least 10 functional human heavy chain V genes are substantially in their natural configuration;
b) at least one human heavy chain D gene and at least one human heavy chain J gene;
c) a murine μ enhancer and switch μ fragment;
d) a murine Cγt fragment which lacks the CH1 exon and
e) a murine 3′ enhancer gene comprising enhancer elements hs3A, hs1.2, hs3B, hs4, hs5, hs6 and hs7.

Purified YAC DNA may be introduced into murine progenitor cells by any method known in the art, including, but not limited to, pronuclear microinjection of freshly fertilised oocytes (K. Becker & B. Jerchow “Generation of Transgenic Mice by Pronuclear Microinjection” pp 99-115 in “Advanced Protocols for Animal Transgenesis. An ISTT Manual.Ed. Shirley Pease & Thomas L. Saunders. Springer Protocols 2011) or transformation of ES cells (VV09305165). Generation of transgenic mice carrying intact YAC DNA on a TKO background may be achieved by a variety of different methods, including introduction of YAC DNA into wild-type (WT) mice followed by backcrossing of transgenic animals onto a TKO background, or direct introduction of the YAC DNA into TKO mice.

HcAbs as referred to herein are heavy chain only antibodies which comprise a human VH domain.

The VH domain expression library may be expressed by any conventional techniques known in the art, for example phage display, ribosome display technology, yeast display, microbial cell display or expression on beads such as microbeads. In one aspect the VH domains are expressed using ribosome display technology (EP0985032; Hanes, J., Pluckthun, A., Proc. Natl. Acad. Sci. USA; 1997; 94(10); 4937-4942; Irving, R A et al, J. Immunol. Methods; 2001; 1; 2489(1-2); 31-45).

Soluble, expressed VH domains may be detected using techniques known in the art, for example immunoblotting, ELISA or by direct purification by affinity chromatography. In one aspect the VH1 domains are detected by immunoblotting.

The sequences of identified soluble VH polypeptides are determined using methods known in the art. The VH1 domain polypeptides identified in step c) comprise diverse CDR3 regions, which therefore need to be removed in order to determine the sequence of the soluble VH1 scaffold (essentially the FR1, CDR1, FR2, CDR2 and FR3 regions).

Libraries

According to a further aspect of the invention there is provided a human VH domain expression library derived from the scaffold of the invention. The library comprises a population of VH clones having at least 70% solubility, is highly expressed, functional and non-aggregating. The library has the advantage of providing for direct and efficient isolation of VH domain antibodies.

In one embodiment there is provided human VH domain expression library derived from the scaffolds according to Seq ID No. 1 and Seq ID No. 2

According to a further embodiment of the invention there is provided a method of constructing a VH domain expression library comprising the steps of;
a) Assembling the scaffold according to the previous aspects with a plurality of CDR3 nucleic acid sequences to obtain a VH domain repertoire
b) Expressing the VH domain repertoire to produce a VH domain library and selecting for functional VH domains against target antigen.

In a further embodiment there is provided a method of constructing a VH domain expression library comprising the steps of;
a) Assembling the scaffold according to Seq ID No. 1 or Seq ID No. 2, comprising CDR3 nucleic acid sequences to obtain a VH domain repertoire,
b) Expressing the VH domain repertoire to produce a VH domain library and selecting for functional VH domains against target antigen.

The method may comprise an additional modification step, for example CDR3 mutagenesis followed by further rounds of screening against target antigen. This may improve VH domain characteristics such as solubility, affinity and immunogenicity.

The method may comprise the additional step of sequencing the selected VH domains.

The method may further comprise the additional step of expressing the selected VH domain in a host cell. Typical examples of host cells include E. coli in particular TG1, BL21(DE3), W3110 and BL21(DE3)pLysS.

The VH domain repertoire may be expressed by any known method in the art, for example phage display or ribosome display as described herein.

The library comprises the VH domain scaffold and enables VH domains which have the advantageous properties of the scaffold including solubility, stability and functionality to be obtained.

In one aspect the invention provides a VH domain library comprising the scaffold sequence according to Seq ID No. 1.

CDR3 regions are known to have the most variability in comparison with CDR1 and CDR2 domains and therefore enable the generation of a library containing at least 10⁹ or more unique VH domains with a common structural framework or scaffold. In a further embodiment the invention comprises libraries comprising at least 10⁹, 10¹⁰, 10¹¹ or 10¹² unique VH domains.

The CDR3 region to be introduced may be derived from any source including human, non-human, synthetic and humanised. CDR3 regions are known to vary in size and typically are between 4 to 25 amino acid residues in length. Typically a CDR3 region is approximately 12 amino acids in length. A humanised antibody repertoire comprises antibodies which are derived from a non-human source and have been modified by the mutation of certain amino acid residues to make the antibody more human-like, for example to impart low immunogenicity characteristics. The number of amino acid residues mutated may vary depending on the desired characteristics. In one embodiment the CDR3 region is derived from a naïve or non-immunized source and may be human, humanised or non-human. A naïve repertoire or library is derived from a source where the animal has not been exposed to antigen. In one example the CDR3 region is derived from a camelid or mouse naïve repertoire. In one example the CDR3 region is human and derived from a naïve repertoire for example peripheral blood lymphocytes, spleen, lymph node, peripheral blood or bone marrow. In a further example the CDR3 region is synthetic or humanised.

The CDR3 region to be introduced may be derived from an immunised source. An immunised repertoire derived from a human or non-human animal which has been exposed to antigen and as a result the repertoire contains antibodies that recognise the antigen. In one example the CDR3 region is derived from a camelid or mouse immunised repertoire. In a further example the CDR3 region is derived from a human immunised repertoire, for example from peripheral blood lymphocytes, spleen, lymph node or bone marrow.

The CDR3 regions may be obtained from commercially available cDNA libraries.

The CDR3 regions may be introduced into the VH scaffold by any suitable method known in the art for example PCR (polymerase chain reaction)-based assembly and amplification using primers overlapping the framework and CDR3 regions. The VH scaffold containing CDR3 regions may be introduced into any suitable vector (for example a phagemid vector) by any suitable method known in the art for example by PCR-based assembly using a mixture of appropriately linearized vector plus DNA encoding VH scaffold containing CDR3 insert followed by PCR amplification using primers overlapping the framework and CDR3 regions. Evaluation of the VH clones is performed for example by ELISA (Enzyme Linked lmmunosorption Assay) following expression using a suitable vector in a host cell, for example E. Coli.

The CDR3 regions may be subject to further mutagenesis after introduction into the scaffolds of the invention. This offers the advantage that the library may be tailored or biased towards a target antigen after an initial round of selection against that antigen to obtain VH domains offering improved affinity, solubility or expression. Alternatively the CDR3 regions may be subject to one or more rounds of mutagenesis prior to selection against antigen. In addition to tailoring the VH library to a particular antigen, further mutagenesis serves to increase the overall size of the repertoire thereby increasing the likelihood of obtaining an antibody with the desired characteristics.

The mutagenesis methods used to introduce further diversity represent general molecular biology techniques known to those skilled in the art including site directed mutagenesis, random mutagenesis, error-prone PCR, insertions and deletions (Ausubel et al, Current Protocols in Molecular Biology, John Wley & Sons, New York 2000).

CDR1, CDR2 and/or CDR3 regions of the VH domains of the invention may comprise one or more acidic amino acids to improve solubility and/or reduce aggregation. Typically the VH domains may comprise Asp or Glu at position 32 of CDR1.

Once the library has been assembled following the introduction of CDR3 regions in a suitable expression vector, the VH domains are expressed for screening against a target antigen. The library may be expressed and screened by any conventional techniques known in the art for example phage display, ribosome display, yeast display, microbial cell display or expression on beads such as microbeads. In one embodiment the library is expressed by any selection display system which permits the nucleic acid of a VH domain to be linked to the expressed VH polypeptide, for example phage display systems wherein VH domains are expressed on the surface of filamentous bacteriophage and screened against target antigen (McCafferty, J., Griffiths, A D., Winter, G., Chiswell, D J, Nature, 348 1990; 552-554). The bacteriophage library may be screened against antigen using techniques well known in the art (for example as described in Antibody Engineering, Edited by Benny Lo, chapter 8, p161-176, 2004) which may be immobilised (for example attached to magnetic beads or on the surface of a microtitre plate) or expressed on the surface of a cell, in solution or in any other format. The skilled person will be aware that the target antigen may be any antigen of interest, for example purified, expressed on the surface of a cell, partially purified or peptides. Typically the target antigen is a purified protein. The library may also be screened against antigen in a high-throughput manner, for example in microarrays. Binding phage are retained, eluted and amplified by infection of E. Coli or other suitable host cells and phage isolated and screened again against target antigen. This process can be repeated numerous times, for example 2 to 10 repeats resulting in the enrichment of VH domains specific for the target antigen or until VH domains possessing the desired characteristics are obtained. The gene sequence encoding the VH domain may then be determined using standard techniques for example amplifying the VH nucleic acid sequence and determining the amino acid sequence, cloning the sequence into an expression vector and expressing in E. Coli, or other suitable host cells to further determine the properties of the isolated VH domain.

Alternatively the VH domain library may be expressed by ribosome display technology wherein the VH are displayed as polypeptides on the surface of a ribosome together with the corresponding mRNA. The ribosome display library may be screened against immobilised antigen (for example attached to magnetic beads or on the surface of a microtitre plate, or using affinity chromatography column with a resin bed containing the ligand). Elution of the binders and dissociation of the mRNA allows for reverse transcription of the mRNA to form the cDNA from which the library was derived. The isolated sequence may then undergo mutagenesis or further rounds of screening in the ribosome display system. The techniques for construction of ribosome display libraries and methods of isolation of antigen binders is well known in the art (EP0985032; Hanes, J., Pluckthun, A., Proc. Natl. Acad. Sci. USA; 1997; 94(10): 4937-4942; Irving, R A et al, J Immunol. Methods; 2001; 1;248(1-2): 31-45).

The invention further provides isolated human VH domains or fragments thereof comprising a scaffold as defined in the previous aspects.

In one embodiment the VH domain antibodies or fragments thereof are characterised in that they comprise the scaffold sequences as defined herein in accordance with Seq ID No. 1 or Seq ID No. 2.

In a further embodiment the VH domain antibodies or fragments thereof comprise a VH scaffold having at least 80%, 90%, 95% or 98% amino acid sequence identity with the sequences according to Seq ID No. 1.

The invention encompasses nucleic acids encoding the VH domain antibodies of the invention. The nucleic acid may be double stranded, single stranded, including cDNA or RNA.

The invention also relates to vectors and host cells comprising the nucleic acid sequences encoding the VH domain of the invention. Suitable vectors are known to those skilled in the art. and include pGEX, pDEST, pET, pRSET, pBAD and pQE. Suitable host cells may be eukaryotic or prokaryotic. Preferably the host cells are bacterial for example E. Coli. Strains of E. Coli known to the skilled person include TG1, BL21(DE3), W3110 and BL21(DE3)pLysS.

The proportion of VH domains in the library of the present invention with improved solubility characteristics may be higher compared to similar libraries of the prior art derived from scaffolds with lower solubility characteristics. The inventors have determined that the proportion of soluble clones present in the library described herein is at least 70%.

The VH domains or fragments thereof may be isolated and purified from the host cells expressing them by techniques known in the art. Purification of VH domains as referred to herein may be carried out by suitable methods known in the art. For example the VH domains may be purified from the host cell or cell culture medium by chromatography, ion-exchange chromatography, size exclusion chromatography, high performance liquid chromatography (HPLC) and affinity chromatography (Methods in Enzymology, Vol. 182, Guide to Protein Purification, Eds. J. Abelson, M. Simon, Academic Press, 1st edition, 1990).

Further to purification, the VH domain may undergo genetic modifications such as mutagenesis in one or more of the CDR regions using standard techniques to improve affinity, solubility or expression, for example site-directed mutagenesis, random mutagenesis, insertions or deletions. If the library is derived from a non-human source then the VH domain may require “humanising” to reduce potential immunogenicity reactions when administered in human therapy. In this respect defined amino acid residues are mutated to engineer the VH domain so that it retains binding affinity and conservative non-human residues are substituted.

The VH domains may form multimers comprising two or more VH domains which is known to improve the strength of binding to antigen by virtue of the increased number of antigen binding sites. For example the VH domains may form homodimers, heterodimers, heteromultimers or homomultimers.

The VH domains may be joined to a moiety designed to optimise the PK/PD characteristics of the VH in systemic circulation. In one example the VH domain may be fused directly to the additional moiety and in another example the VH domain may be coupled chemically to the additional moiety either directly or via a linker. The linker may comprise a peptide, an oligopeptide, or polypeptide, any of which may comprise natural or unnatural amino acids. In another example, the linker may comprise a synthetic linker. In one example the additional moiety may be a naturally occurring component (for example serum albumin) or in another example the additional moiety may be polyethylene glycol.

The VH domains may be joined to a toxic moiety with the aim of utilising the binding of the VH domain to its target antigen in vivo to deliver the toxic moiety to an extracellular or intracellular location. The toxic moiety may be fused directly to the VH domain and in another example the toxic moiety may be coupled chemically to the VH domain either directly or via a linker. The linker may comprise a peptide, an oligopeptide, or polypeptide, any of which may comprise natural or unnatural amino acids. In another example, the linker may comprise a synthetic linker.

Further to isolation of the VH domain in accordance with known techniques and as described above, the VH domain may be assayed to determine affinity for the target antigen. This may be carried out by a number of techniques known in the art for example enzyme-linked immunospecific assay (ELISA) and BIAcore (measurement surface plasmon resonance in real time reactions between molecules). In addition, binding to cell surface antigens can be measured by fluorescence activated cell sorting (FACS). The affinity of the isolated VH domain indicates the strength of binding to the target antigen and is a crucial parameter in determining whether a candidate VH domain is likely to proceed further into developments as a therapeutic. Affinity is commonly measured by the dissociation constant K_{d} (K_{d}=[antibody][antigen]/[antibody/antigen complex]) in molar (M) units. A high K_{d} value represents an antibody which has a relatively low affinity for a target antigen. Conversely a low K_{d}, often in the sub-nanomolar (nM) range indicates a high affinity antibody.

In a further aspect the invention provides a pharmaceutical composition comprising a therapeutically effective amount of a VH antibody derived from the VH libraries of the invention, and a pharmaceutically acceptable excipient. VH antibodies derived from the libraries of the invention possess the desirable characteristics of high solubility, low propensity to aggregate, stability and functionality. Such characteristics allow the VH antibodies to be progressed for therapeutic development and use as diagnostics without the requirement for substantial engineering or modification. The invention provides pharmaceutical compositions comprising a VH domain in an effective amount for binding to a target antigen and a pharmaceutically acceptable excipient. Suitable pharmaceutically acceptable excipients are known to those skilled in the art and generally includes an acceptable composition, material, carrier, diluent or vehicle suitable for administering the VH domains of the invention to an animal. In this respect the VH domain may be comprised in a whole antibody or fragment thereof. For example the VH domain may be grafted onto a human antibody framework, for example an IgG using methods known in the art.

In a further embodiment the invention provides a method of treatment by administering an effective amount of the VH domain of the present invention to an animal. In this respect the VH domain may be comprised in a whole antibody or fragment thereof. For example the VH domain may be grafted onto a human antibody framework, for example an IgG using methods known in the art.

The invention is described further in the following non-limiting examples.

### EXAMPLES

### Example 1

### Construction of YACs

BACs (bacterial artificial chromosomes in a circular format) are tools well known in the art that facilitate the manipulation (e.g. sequencing and cloning) of segments of DNA from ˜150 kbp-350 kbp in size (Methods in Molecular Biology, Volume 54 and 349). BACs containing DNA derived from the heavy chain immunoglobulin locus of humans or mice are numerous and well known in the art. Examples of such BACs include but are not limited to:

Human:
RP11-1065N8
RP11-659B19
RP11-14117
RP11-72N10
RP11-683L4
RP11-12F16

Murine:
RP23-354L16
RP24-72M1

It is also well known in the art that BACs can be used to facilitate the sequential molecular joining of multiple DNA segments comprising overlapping (complementary) sequence to create much larger DNA molecules (e.g. YACs). One such method for achieving this, BIT (bridge induced translocation), requiring the linearization of BACs to create yeast artificial chromosomes (YACs) is described in YAC Protocols (Methods in Molecular Biology, Volume 54 and 349).

Such heavy chain YACs (HC YACs) may comprise a number of human VH genes, human D genes, human J genes, at least one C-region (which may be of either human or murine origin) lacking the CH1 domain (deleted using standard molecular biology techniques well known in the art) and key regulatory elements such as enhancer and switch elements (also known in the art).

### Example 2

### Identification of Soluble VH1-02 Scaffold from Transgenic Mice Expressing Human VH Fragments

Transgenic mice (subsequently called HC transgenic mice) devoid of functional endogenous murine immunoglobulin loci and comprising a HC YAC (see example 1) were generated These mice are unable to produce endogenous murine immunoglobulin and instead express heavy chain-only antibodies (HcAbs) devoid of light chains and which comprise human VH domains matured in the absence of a partner VL domain. Only B cells expressing soluble HcAbs (which therefore comprise soluble VH domains) are expanded in these mice. Studies were performed to identify which germline families derived from the integrated YAC construct were preferentially used for VH production.

To analyse the expression of human VH fragments from HC transgenic mice, RT-PCR cloning was employed to amplify VH genes expressed by B cells from samples of lymphoid tissue derived from blood and spleen. RNA was isolated from the lymphoid samples using commercially available reagents (e.g. RNeasy Qiagen kit cat no. 74106), and VH mRNA reverse transcribed using primer Human CH2 rev (Table 1) and Superscript III enzyme (Gibco), using associated buffers and conditions as recommended by the manufacturer. The cDNA was then used in PCR reactions to amplify VH regions, using germline-specific primers (Table 1).

TABLE 1
Oligonucleotide primers (5′ to 3′)

| | | Seq |
|---|---|---|
| Primer | Sequence | ID No |
| Human CH2 rev | GTCCGGGAGATCATGAGAGTG | 9 |
| V1/B | GGAACAGACCACCATGGCCCAGGTBCAGCTGGTGCAGTCTGG | 10 |
| V2/B | GGAACAGACCACCATGGCCCAGATCACCTTGAAGGAGTCTGG | 11 |
| V4-4/B | GGAACAGACCACCATGGCCCAGGTGCAGCTGCAGGAGTCGGG | 12 |
| V6/B | GGAACAGACCACCATGGCCCAGGTACAGCTGCAGCAGTCAGG | 13 |
| VH_J/F | GCTACCGCCACCCTCGAGTGARGAGACRGTGACC | 14 |
| pHENAPmut4 | GTCCATGGCCATCGCCGGCTGGGCCGCGAG | 15 |
| pHENAPmut5 | TAGCAGCCTCGAGGGTGGCGGTAGCCATCACCACCATCACCACGGGAGC | 16 |
| V1a/B | GGAACAGACCACCATGGCCCAGGTBCAGCTGGTGCAGTCTGGGGCTGAGG | 17 |
| VHCDR3/B/G- | GACACGGCCGTGTATTACTGTGC | 18 |
| VHCDR3/F/C- | GCACAGTAATACACGGCCGTGTC | 19 |

PCR reactions were performed using Phusion high fidelity DNA polymerase, (Finnzymes F-531L) and “touch-down” PCR cycling conditions.

Example touchdown programme;

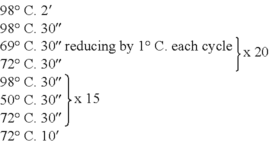

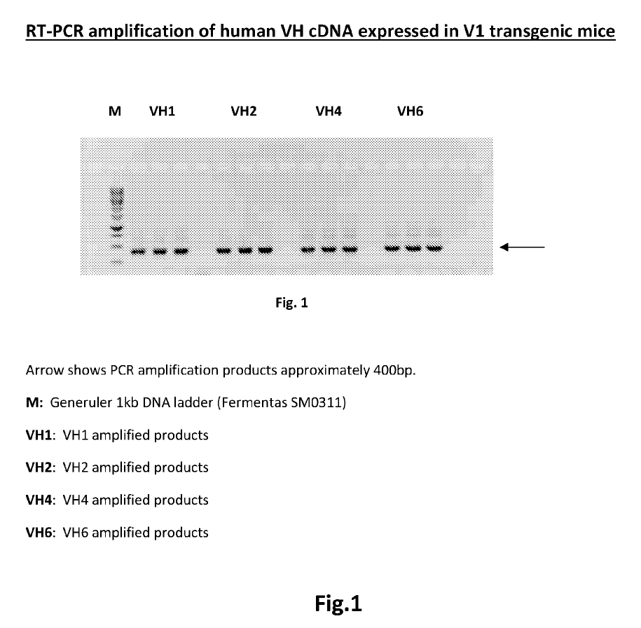

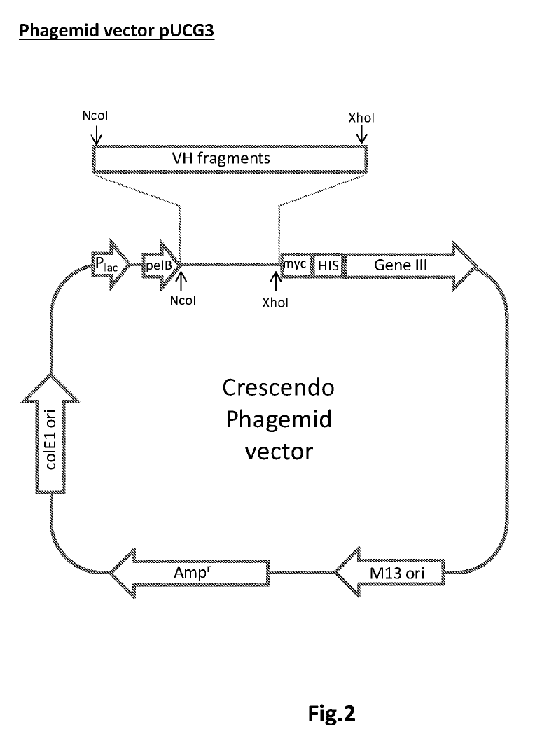

PCR products generated for human VH present in the HC YAC construct (FIG. 1 were purified and then cloned into phagemid vector pUCG3 for expression (FIG. 2).

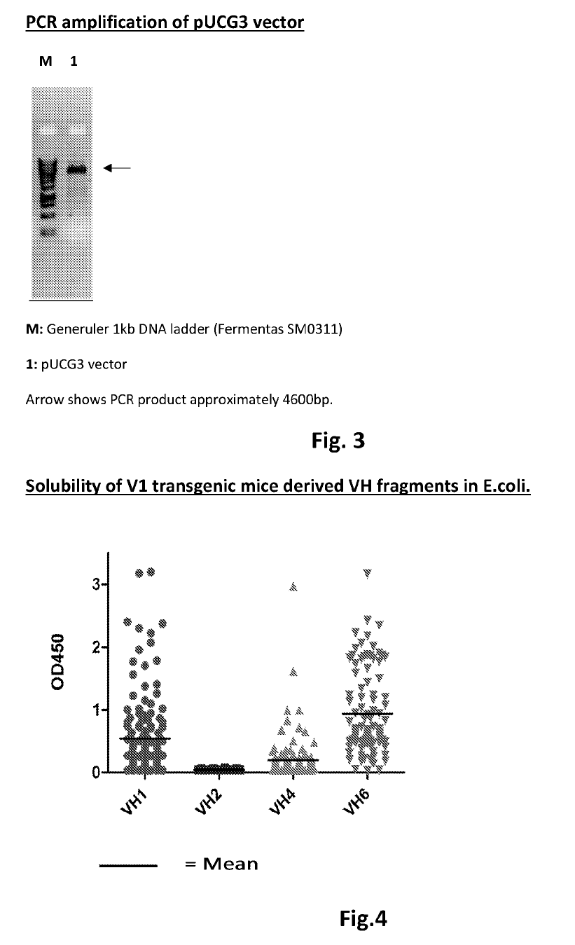

pUCG3 DNA for cloning was prepared by PCR as follows: 1000 ul 2× Phusion PCR mix; 60 ul of oligonucleotide pHENAPmut4 (16 uM); 60 ul of oligonucleotide pHENAPmut5 (16 uM); 400 ng of pUCG3 miniprep DNA and dH₂O to 2000 ul final volume. The reaction was divided equally into 40 tubes and then heated to 95° C. for 1 minute followed by 30 cycles of PCR: 98° C. 10 seconds, 72° C. 2 minutes. After 30 cycles the PCR reactions were then heated at 72° C. for 5 minutes followed by holding at 10° C. Products of PCR were then analysed by electrophoresis on 1% (w/v) agarose gels followed by staining with ethidium bromide. PCR products were observed at the expected size of approximately 4600 bp (FIG. 3). The PCR product was purified using Fermentas PCR purification columns (K0701) and resuspended in dH₂O.

Both the pUCG3 vector preparation and purified VH RT-PCR products from HC transgenic mice were digested with Ncol (Fermentas FD0574) and Xhol (Fermentas FD0694) restriction enzymes overnight at 37° C. The pUCG3 restriction digest only was then incubated with shrimp alkaline phosphatase for 4 hours at 37° C. according to the manufacturers instructions (Fermentas EF0511). All digests were heated to 80° C. for 5 minutes and then each product purified using Fermentas PCR purification columns (K0701) and finally resuspended in dH₂O.

The digested VH products from HC transgenic mice were ligated into pUCG3 using NEB T4 DNA ligase (M0202M) following the manufacturers instructions. Briefly, Ncol/Xhol double-digested pUCG3 DNA and VH products were mixed at a molar ratio of 1:2 and incubated with T4 ligase for 4 hours at 16° C. Following incubation at 70° C. for 30 minutes, the products of ligation were transformed into E.coli strain TG1 using standard chemical transformation techniques (Walhout et al., Methods Enzymol; 328: p575-92, 2000).

Expression of VH Fragments in E. coli

Following the cloning of different VH germline genes into pUCG3, the expression and solubility of VH fragments produced by HC transgenic mice were investigated by analysis of bacterial periplasmic extracts. Following cloning into pUCG3 all VH fragments included at their N-terminus a pelb leader sequence that directed them to the periplasmic space following expression. VH fragments that are insoluble or aggregated accumulate in the cytoplasm as inclusion bodies, thus, only soluble VH fragments cross the bacterial membrane into the periplasm, Therefore, ELISA-based detection of VH fragments in bacterial periplasmic extracts was considered a good surrogate measure of VH solubility. More than 90 individual colonies from each VH germline family cloned into pUCG3 were picked into wells of a Nunc 96 deep well plate containing 1000 ul per well of 2XYT broth supplemented with 2% (w/v) glucose and 100 ug/ml ampicillin.

The plates were then grown at 37° C. with shaking at 250 rpm for 5-6 hours. Plates were centrifuged at 3200 rpm for 10 mins and the supernatant discarded. Cell pellets were then resuspended in 1 ml 2XYT containing 100 ug/ml ampicillin and 1 mM IPTG, and the plates incubated overnight at 30° C. with shaking at 250 rpm. Plates were centrifuged at 3200 rpm for 10 mins and the cell pellets resuspended in 80 ul of sucrose buffer (20% sucrose, Babraham Stores 101361, 1 mM EDTA, Sigma E5134, 50 mM Tris-HCl pH 8, Melford 1185-53-1), and then placed on ice for 30 mins. The plates were then centrifuged at 4500 rpm for 15 mins and 50 ul of supernatant from each well transferred to the corresponding well of a Nunc 96 well maxisorb plate (Nunc 443404). This supernatant, the bacterial periplasmic extract (containing any soluble expressed VH), was then incubated for 2 hours at room temperature to coat proteins to the plate.

The wells of the Nunc plates were then washed once with PBS buffer and then 200 ul per well of 3% (w/v) Marvel PBS added. Plates were then incubated for 1 hour at room temperature. The wells of the Nunc plates were again washed once with PBS buffer and then 50 ul per well of HRP-conjugated anti-HIS monoclonal antibody (Miltenyi Biotech, 130-092-7853%), diluted 1:1000 in 3% (w/v) Marvel PBS added. Plates were then incubated for a further 1 hour at room temperature. The wells of the Nunc plates were then washed three times with PBST buffer followed by three washes with PBS buffer, and then to each well added 50 ul of TMB developer (Sigma T0440). Plates were incubated for up to 10 minutes and then TMB development was stopped by the addition of 25 ul per well of 0.5M sulphuric acid solution.

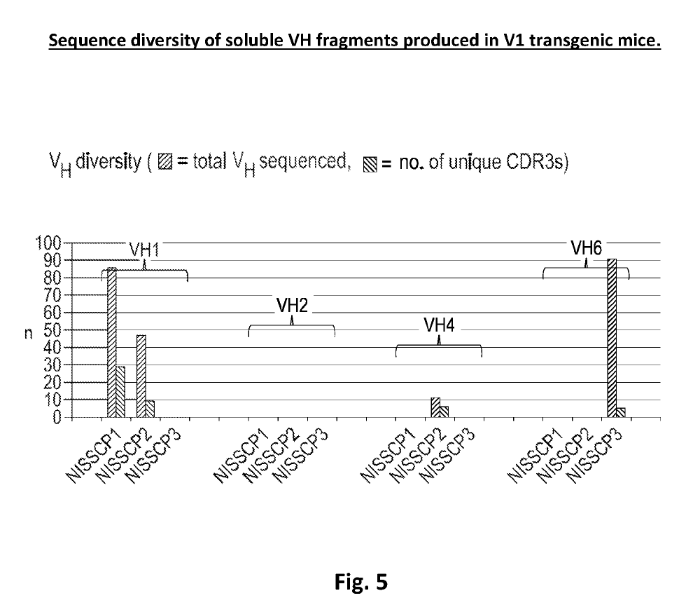

Plates were then read on the Biorad iMark plate reader to measure the absorbance at 450 nm in each well. Solubility results were then plotted on graphs and the VH1 and VH6 families were shown to produce a large number of soluble VH (FIG. 4). DNA sequencing was performed to determine the level of VH diversity within the soluble VH1, VH4 and VH6 populations and the highest CDR3 diversity was observed in the VH1 family (FIG. 5). Therefore this family, human germline VH1-02, was chosen as a soluble scaffold on which to build phage display libraries for human VH discovery.

### Example 3

### Analysis of VH1-02 Solubility, Expression, Stability and Aggregation

To further demonstrate favourable biophysical properties of the VH1-02 framework, two VH antibodies (7D7, Seq ID No. 3 and Seq ID No. 4 and 6B2, Seq ID No. 5 and Seq ID No. 6) each derived from HC transgenic mice and both of germline VH1-02 sequence, were expressed and purified from 50 ml shake flask cultures. Each VH protein has a C-terminal 6×HIS tag that enabled purification from bacterial periplasmic extracts by nickel-agarose affinity chromatography.

A starter culture of each VH was grown overnight in 5 ml 2XTY broth (Melford, M2103) supplemented with 2% (w/v) glucose+100 ug/ml ampicillin at 30° C. with 250 rpm shaking. 50 ul of this overnight culture was then used to inoculate 50 ml 2XTY supplemented with 2% (w/v) glucose+100 ug/ml ampicillin and incubated at 37° C. with 250 rpm shaking for approximately 6-8 hours (until OD600 =0.6-1.0). Cultures were then centrifuged at 3200 rpm for 10 mins and the cell pellets resuspended in 50 ml fresh 2XTY broth containing 100 ug/ml ampicillin+1 mM IPTG. Shake flasks were then incubated overnight at 30° C. and 250 rpm. Cultures were again centrifuged at 3200 rpm for 10 mins and supernatants discarded. Cell pellets were resuspended in 1 ml ice cold extraction buffer (20% (w/v) sucrose, 1 mM EDTA & 50 mM Tris-HCl pH8.0) by gently pipetting and then a further 1.5 ml of 1:5 diluted ice cold extraction buffer added. Cells were incubated on ice for 30 minutes and then centrifuged at 4500 rpm for 15 mins at 4° C. Supernatants were transferred to 50 ml Falcon tubes containing imidazole (Sigma, 12399—final concentration 10 mM) and 0.5 ml of nickel agarose beads (Qiagen, Ni-NTA 50% soln, 30210) pre-equilibrated with PBS buffer. VH binding to the nickel agarose beads was allowed to proceed for 2 hours at 4° C. with gentle shaking. The nickel agarose beads were then transferred to a polyprep column (BioRad, 731-1550) and the supernatant discarded by gravity flow. The columns were then washed 3 times with 5 ml of PBS+0.05% Tween followed by 3 washes with 5 ml of PBS containing imidazole at a concentration of 20 mM. VH were then eluted from the columns by the addition of 250 ul of PBS containing imidazole at a concentration of 250 mM. Imidazole was then removed from the purified VH preparations by buffer exchange with NAP-5 columns (GE Healthcare, 17-0853-01) and then eluting with 1 ml of HBS-EP buffer (Biacore, BR-1006-60). Yields of purified VH were 3.2 mg/litre for 7D7 and 30 mg/litre for 6B2.

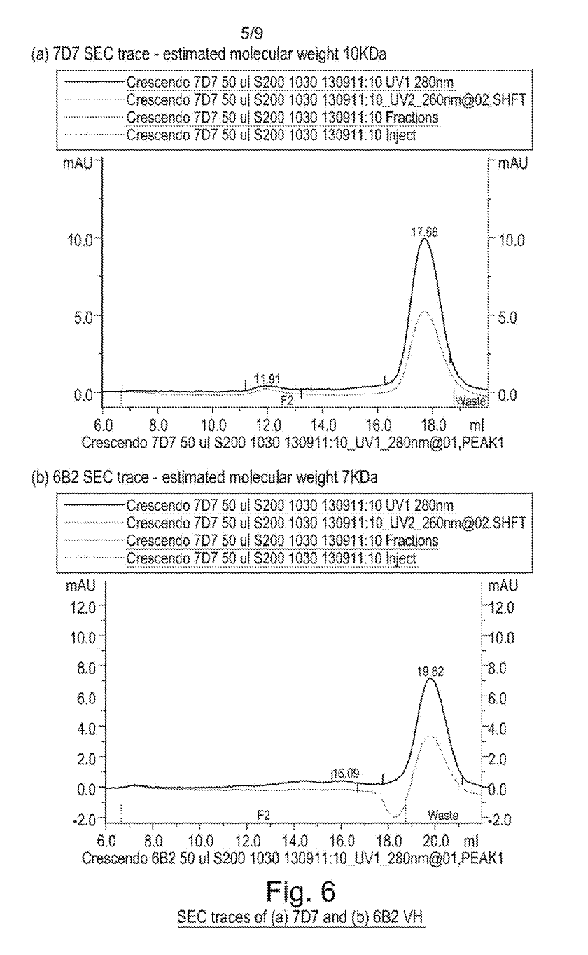

VH stability and aggregation was determined by SEC (size exclusion chromatography) using the Akta Explorer FPLC and a Superdex 200 10/30 HR column (GE lifesciences). 7D7 and 6B2 VH samples were diluted to 200 ug/ml in HBS-EP buffer and centrifuged at 18000×g for 10 min 4° C. 50 ul of VH was then injected onto the Superdex column and elution monitored by absorbance at 280 nm. SEC traces for 7D7 and 6B2 VH are presented in FIG. 6. Molecular weights were determined by comparison with the elution profiles of known standards.

### Example 4

### Methods for Preparation of CDR3 Domains

Human cDNA from spleen, lymph node, bone marrow and peripheral blood lymphocytes was purchased from commercial sources (Invitrogen, Clontech). Oligonucleotide primers VHCDR3/B/G- and VHJ/F were synthesised to facilitate PCR amplification of VH-CDR3 plus VH framework 4 sequences from B cell cDNA.

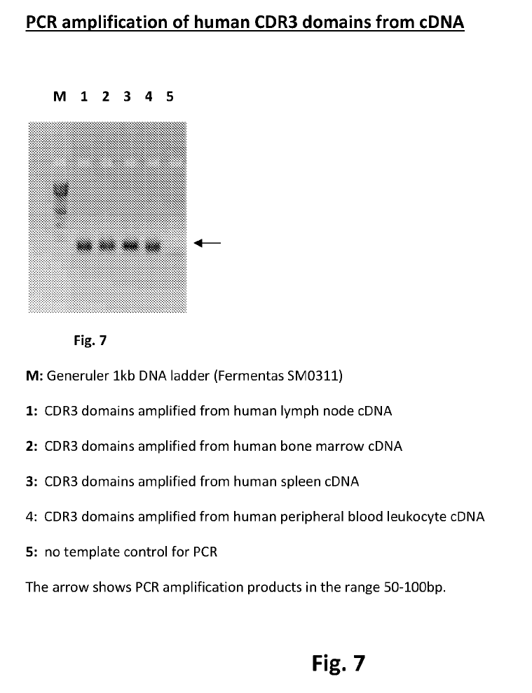

Individual PCR reactions were set up for each cDNA sample as follows: 25 ul 2× Phusion PCR mix (Finnzymes F-531L); 2.5 ul VHCDR3/B/G- (10 uM); 2.5 ul VHJ/F (10uM); 3 ng cDNA and dH₂O to 50 ul final. Reactions were then heated to 95° C. for 1 minute followed by 30 cycles of PCR: 98° C. 10 seconds, 54° C. 30 seconds, 72° C. 30 seconds. After 30 cycles PCR reactions were then heated at 72° C. for 8 minutes followed by holding at 10° C. Products of PCR were then analysed by electrophoresis on 1% (w/v) agarose gels followed by staining with ethidium bromide. PCR amplification products were observed at the correct size of approximately 50-100 bp (FIG. 7).

### Example 5

### Library Assembly

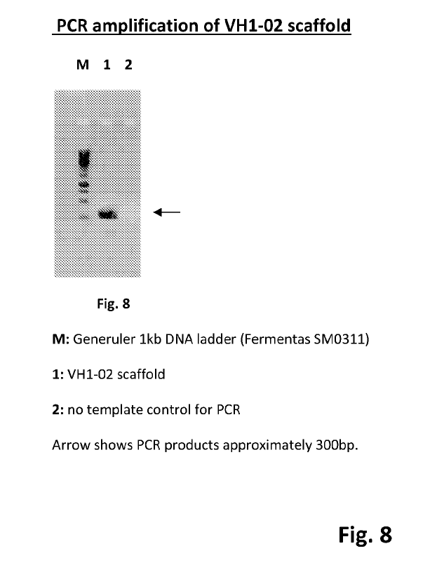

The VH1-02 scaffold was amplified by PCR (Finnzymes F-531L) as follows: 25 ul 2× Phusion PCR mix; 2.5 ul V1a/B (10 uM); 2.5 ul VH3-93/F/C- (10 uM); 10 ng of plasmid encoding VH1-02 and dH₂O to 50 ul final. Reactions were then heated to 95° C. for 1 minute followed by 30 cycles of PCR: 98° C. 10 seconds, 54° C. 30 seconds, 72° C. 30 seconds. Products of PCR were then analysed by electrophoresis on 1% (w/v) agarose gels followed by staining with ethidium bromide. PCR amplification products were observed at the correct size of approximately 300 bp (FIG. 8).

Human VH-CDR3 PCR products (Example 3) were then assembled with the VH1-02 scaffold to generate DNA products encoding full length VH antibodies. The VH1-02 scaffold was assembled with amplified human VH-CDR3 sequences by adding the following: 12.5 ul 2× Phusion PCR mix (Finnzymes F-531L); 40 ng of VH1-02 PCR product; 10 ng of each VH-CDR3 PCR product (Example 3) and dH₂O to 25 ul final. The reaction was then heated to 95° C. for 1 minute followed by 8 cycles of PCR: 98° C. 10 seconds, 54° C. 30 seconds, 72° C. 30 seconds. After 8 cycles PCR reactions were then heated at 72° C. for 8 minutes followed by holding at 10° C.

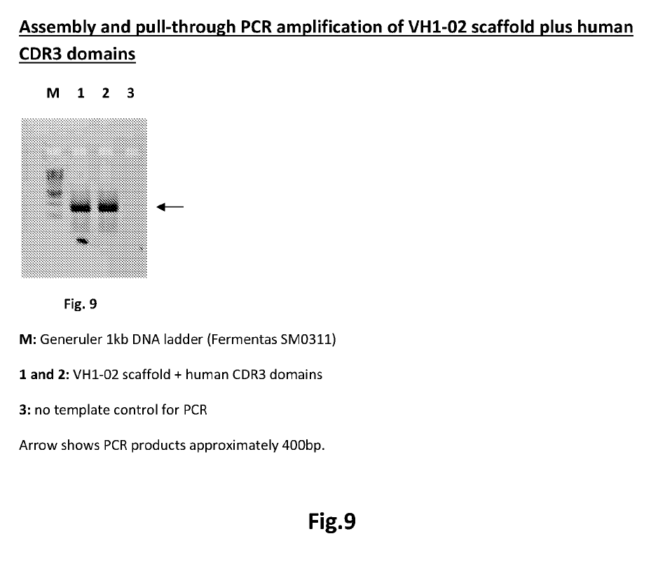

Full-length VH products were then amplified from the assembly products by pull-through PCR: 100 ul 2× Phusion PCR mix (Finnzymes F-531L); 10 ul of oligonucleotide V1a/B (10 uM); 10 ul of oligonucleotide VHJ/F (10 uM); 10 ul of VH1-02 assembly products and dH₂O to 200 ul final volume. Reactions were then heated to 95° C. for 1 minute followed by 30 cycles of PCR: 98° C. 10 seconds, 54° C. 30 seconds, 72° C. 30 seconds. After 30 cycles PCR reactions were then heated at 72° C. for 8 minutes followed by holding at 10° C. Products of PCR were then analysed by electrophoresis on 1% (w/v) agarose gels followed by staining with ethidium bromide. Full length VH products were observed at the expected size of approximately 400 bp (FIG. 9). The PCR products were purified using Fermentas PCR purification columns (K0701) and resuspended in dH₂O.

To prepare libraries for phage display, full-length VH products were cloned into phagemid vector pUCG3 (FIG. 2). Preparation of pUCG3 DNA for cloning was described in Example 1. The VH1-02 pull-through PCR products were digested with Ncol (Fermentas FD0574) and Xhol (Fermentas FD0694) restriction enzymes overnight at 37° C. All digests were heated to 80° C. for 5 minutes and then each product purified using Fermentas PCR purification columns (K0701) and finally resuspended in dH₂O.

The digested VH products were ligated into pUCG3 using NEB T4 DNA ligase (M0202M) following the manufacturers instructions. Briefly, Ncol/Xhol double-digested pUCG3 DNA and VH products were mixed at a molar ratio of 1:2 and incubated overnight with T4 ligase at 16° C. Following incubation at 70° C. for 30 minutes, the products of ligation were purified using using Fermentas PCR purification columns and finally resuspended in dH₂O. Then, using Biorad cuvettes (165-2089) and a Biorad Micropulser, 2 ul of the purified ligation products were electroporated into 25 ul of electrocompetent TG1 cells (Lucigen 60502-1) following the manufacturer's instructions. Electroporated TG1 cells were plated onto 2×TY agar plates supplemented with ampicillin at 100 ug/ml and glucose at 20% (w/v) and incubated overnight at 30° C. Also a dilution series of electroporated TG1 cells were plated to determine library size. The library size was calculated to be 7.7e9 recombinants for the VH1-02 library. Successful library construction was confirmed by sequencing analysis, revealing that 95% of VH possessed unique CDR3 sequences of between 5 and 24 amino acids in length.

### Example 6

### Analysis of Library Composition to Determine the Proportion of Soluble Clones

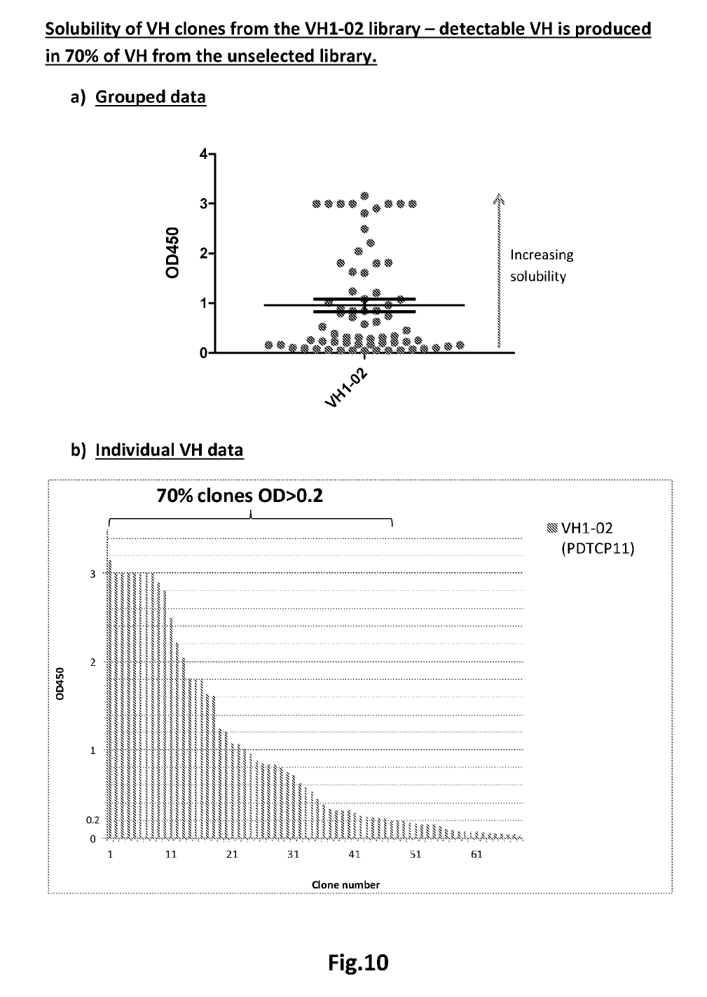

The solubility of over 90 individual VH fragments produced from the VH1-02 library was investigated by analysis of bacterial periplasmic extracts, following the method described in example 1. Solubility results were then plotted on graphs (FIG. 10a and 10b) showing 70% of the clones had an OD greater than or equal to 0.2 at 450 nm.

### Example 7

### Screening Libraries Against Antigen

Phage display-based selection using the VH1-02 library was used to generate VH antibodies that bind to protein and peptide antigens. Preparation of library phage stocks and phage display selections were performed according to published methods (Antibody Engineering, Edited by Benny Lo, chapter 8, p161-176, 2004). Selections were performed on human TNF-α (Gift from Andreas Hoffmann, Martin-Luther-Universitat Halle-Wittenberg), biotinylated beta-amyloid peptide (Bachem H-5642) and a soluble cytokine. TNF-α and the soluble cytokine were immobilised onto maxisorb plates (Nunc 443404) at 10 ug/ml in PBS buffer. For biotinylated beta-amyloid peptide, neutravidin was first immobilised onto maxisorb plates at 10 ug/ml in sodium carbonate buffer, and then used to capture biotinylated beta-amyloid diluted at 5 ug/ml in PBS buffer. For each immobilised antigen, two rounds of phage display selection were performed.

### Example 8

### Analysis of Isolated VH Domains and Sequencing

Following selections of the VH1-02 library on TNF-α, beta-amyloid and a soluble cytokine, specific VH antibodies were identified by phage ELISA following published methods (Antibody Engineering, Edited by Benny Lo, chapter 8, p161-176, 2004). Phage ELISAs were performed against each target protein and an unrelated antigen as control. DNA sequencing of VH antibodies binding specifically to target protein was performed to analyse diversity of VH produced (Table 2 and Table 3), and the output was found have expected levels of diversity.

TABLE 2
Summary of VH isolated to human TNF-α,
β-amyloid and a soluble cytokine.

| | Colonies | Specific VH by | Number | |
|---|---|---|---|---|
| Antigen | picked | ELISA | sequenced | Unique VH |
| TNF-α | 90 | 78 | 78 | 16 |
| B-amyloid | 90 | 83 | 83 | 55 |
| Soluble cytokine | 651 | 292 | 80 | 22 |

TABLE 3
CDR3 sequences of VH isolated to
human TNF-α and beta amyloid peptide.

| | | | Seq |
|---|---|---|---|
| Antigen | VH germline | CDR3 sequence | ID No |
| TNF-α | 1-02 | AARPGTLYYFHEIDV | 20 |
| | | APGWVDGRYFHQLEN | 21 |
| | | APIGAVPTAFNIFYYHYMDV | 22 |
| | | DPGGYYNPRQETFYYYHHMDV | 23 |
| | | GAFDYGDPPPFHYMDV | 24 |
| | | GGLDDSGYPRLLPHYFDY | 25 |
| | | GTLSSGYYYHYFDY | 26 |
| | | LPKDYNAYQYGLDV | 27 |
| | | LPKDYNSYQYGLDV | 28 |
| | | LPKYYNSYQYGLDV | 29 |
| | | LPRTYYDNTGAFDQ | 30 |
| | | MGSNWPHEGYYYHYMDV | 31 |
| | | QEGLVDSYYGMDV | 32 |
| | | RPLGYCGDGSCPFDY | 33 |
| | | SIAATRPNYA | 34 |
| | | TSGSGNYLPYHYAMDV | 35 |
| Beta | 1-02 | AFGNY | 36 |
| amyloid | | AKTGLHLGELSQTRVYFDY | 37 |
| | | ARGYTGRVYFDN | 38 |
| | | ARSERGYGRHYFDH | 39 |
| | | ASLGTGFWSGYSRHYFDN | 40 |
| | | AVGEGPYAHADH | 41 |
| | | DDGTTGSRRSLEI | 42 |
| | | DITGDLGRSGYRSYFDY | 43 |
| | | DKGGAYCSGGNCYYPFDY | 44 |
| | | DMSPVIAPLLGKTRYYYVMDV | 45 |
| | | DRDFWSGFYHHRSYFDY | 46 |
| | | DRGGIVGTKVRRHLDY | 47 |
| | | DTRPRTSSRRYFDY | 48 |
| | | EALVSSTRHYFDS | 49 |
| | | EGYSSGPKYFFDY | 50 |
| | | EIFFTSGRRFLDP | 51 |
| | | EIGTSGSYRRYFDS | 52 |
| | | ERDSNYANKHYFDY | 53 |
| | | ERGGPFGAARHYFDY | 54 |
| | | ERSRTPFRRYFDF | 55 |
| | | ERYFDSSTYRRYFDS | 56 |
| | | ESTASPSRRYLDY | 57 |
| | | GAQIASAATRHYFDY | 58 |
| | | GEYDYVRGTYTYFGLDI | 59 |
| | | GFGNV | 60 |
| | | GGASARYYFDY | 61 |
| | | GGQLRVALDN | 62 |
| | | GKHGYATH | 63 |
| | | GKWDGTRYLLVY | 64 |
| | | GMKGYYGSSRRYYFDY | 65 |
| | | GPPYCSDIGCYTAGDY | 66 |
| | | GQIRNWNYGGTLRD | 67 |
| | | GRRSGSGRVYFDN | 68 |
| | | GTGYDSSGYYQRADK | 69 |
| | | GVAARPGRAYLDS | 70 |
| | | GVESVRRRRNGYLDP | 71 |
| | | HRGYSYTSPFDY | 72 |
| | | LHYSNSWTGPSDY | 73 |
| | | MTSKKYILTGHYKGSNYYAMDV | 74 |
| | | PGGP | 75 |
| | | QLTKVAAGRRYFDS | 76 |
| | | RAAHHLTGQPARYPLDV | 77 |
| | | RHV | 78 |
| | | SHYDNVSGYFTSDK | 79 |
| | | SPPAVVKKYYFDY | 80 |
| | | SRTVGRSYPFDY | 81 |
| | | TKEQWGLVGSRVHFDY | 82 |
| | | VASERVTYGAFDI | 83 |
| | | VAVEARPYGGLDI | 84 |
| | | VGRKYFYERSGYNDVFDF | 85 |
| | | VKGGRGRMDV | 86 |
| | | VLGTGTTGPAVY | 87 |
| | | VMGVPVTSTNRPRHIFDL | 88 |
| | | VRDTFGYRHYFDS | 89 |
| | | VRGSGRWGAFDY | 90 |
| | | VSGRGPFGHADY | 91 |

### Example 9

### Analysis of VH Solubility and Expression

VH antibodies from selections on TNF-α were expressed and purified from 50 ml shake flask cultures as described in example 2.

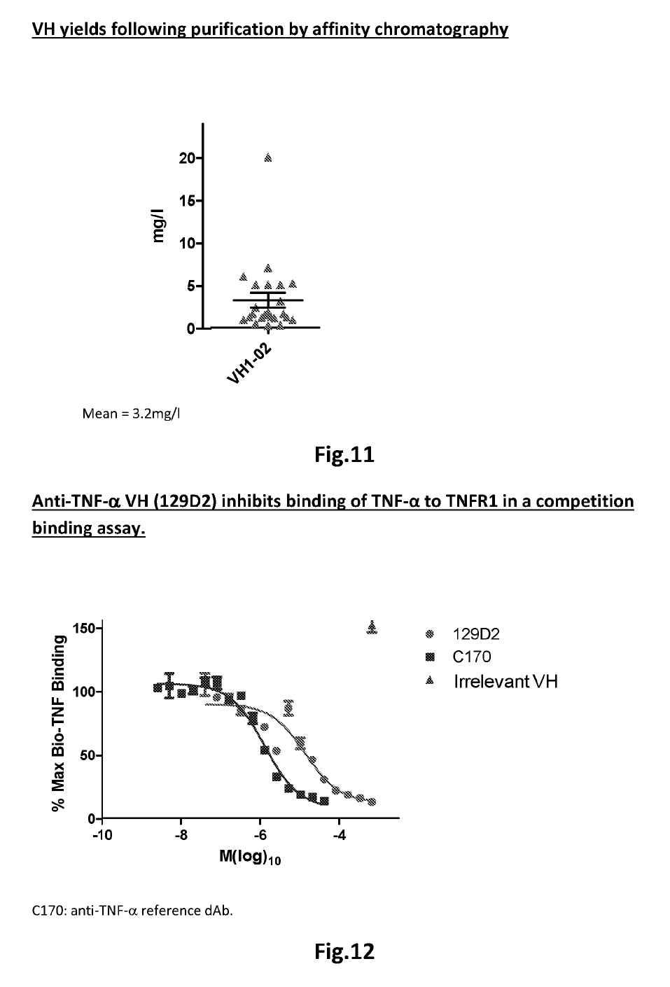

Yields of purified VH from the VH1-02 library are summarised in FIG. 11.

### Example 10

### Anti-TNF-α VH Inhibit Binding of TNF-α to TNFR1 in a Competition Binding Assay

To demonstrate whether anti-TNF-α VH possessed inhibitory properties, a binding assay was developed to measure binding of TNF-α to TNFR1. Inhibitory VH, on binding to TNF-α, blocked TNF-a binding to the receptor and thus reduced the signal observed in the assay. TNFR1 (Sino Biologics, 10872-H03H) was diluted to 0.2 ug/ml (1.8 nM) in PBS and 50u1 per well added to a Nunc maxisorp 96 well plate (Fisher, DIS-071-010P). The plate was then incubated overnight at 4° C. The plate was washed once in PBS, 200 ul per well of blocking buffer (3% marvel in PBS) added and then incubated for 1 hour at room temperature. Dilution series of anti-TNFα VH were prepared in blocking buffer and incubated for 1 hour at room temperature in Greiner plates (650207). The TNFR1 coated maxisorp plate was then washed once with PBS and 40 ul per well of each VH dilution series transferred from the Greiner plate to the corresponding wells of the maxisorp plate. Following incubation for 1 hour at room temperature, 10 ul per well of biotinylated-TNF-α (Gift from Andreas Hoffmann, Martin-Luther-Universitat Halle-Wittenberg) was added to a final concentration of 1 nM and the plate incubated for 1 hour at room temperature. The plate was washed 3 times with PBS Tween and then 3 times with PBS and then 50 ul per well of Neutravidin-HRP (Pierce, 31030) added at a dilution of 1:5000 in blocking buffer. The plate was again incubated for 1 hour at room temperature following which it was washed 3 times with PBS Tween and then 3 times with PBS. Then 50 ul of TMB developer solution (Sigma T0440) was added to each well and the plate allowed to incubate at room temperature until suitable blue colour had developed. Then 50 ul of 0.5M sulphuric acid was added to each well to stop the reaction and absorbance at 450 nm read on a spectrophotometer.

The activity of several anti-TNF-α VH clones were measured in this assay and candidates with inhibitory properties were identified, one example of which (clone 129D2, Seq ID No. 7 and Seq ID No. 8) is shown in FIG. 12. The identification of anti-TNF-α VH antibodies as described herein, with high affinity, antigen specificity, which are also soluble and stable validates the utility of libraries derived from the scaffolds of the invention in the isolation of further VH antibodies to other target antigens with comparable solubility, functionality and stability characteristics.

Scaffold sequences
Seq ID No. 1: VH1-02 amino acid sequence
QVQLVQSGAEVKKPGASVKVSCKASGYTFTGYYMHWVRQAPGQGLEWMG
WINPNSGGTNYAQKFQGRVTMTRDTSISTAYMELSRLRSDDTAVYYCAR
Seq ID No. 2: VH1-02 nucleic acid sequence
CAGGTGCAGCTGGTGCAGTCTGGGGCTGAGGTGAAGAAGCCTGGGGCCT
CAGTGAAGGTCTCCTGCAAGGCTTCTGGATACACCTTCACCGGCTACTA
TATGCACTGGGTGCGACAGGCCCCTGGACAAGGGCTTGAGTGGATGGGA
TGGATCAACCCTAACAGTGGTGGCACAAACTATGCACAGAAGTTTCAGG
GCAGGGTCACCATGACCAGGGACACGTCCATCAGCACAGCCTACATGGA
GCTGAGCAGGCTGAGATCTGACGACACGGCCGTGTATTACTGTGCGAGA
Seq ID No. 3: VH 7D7 amino acid sequence
QVQLVQSGAEVKKPGASVKVSCKASGYTFTGYNMHWVRQAPGQGLEWMG
WINPNSGGTNYAQKFQGRVTMTRDTSISTAYMELSRLRSDDTAVYYCAR
EGSSTVTREMDVWGQGTTVTVSS
Seq ID No. 4: VH 7D7 nucleic acid sequence
CAGGTGCAGCTGGTGCAGTCTGGGGCTGAGGTGAAGAAGCCTGGGGCCT
CAGTGAAGGTCTCCTGCAAGGCTTCTGGATACACCTTCACAGGCTACAA
TATGCACTGGGTGCGACAGGCCCCTGGACAAGGGCTTGAGTGGATGGGA
TGGATCAACCCTAACAGTGGTGGCACAAACTATGCACAGAAGTTTCAGG
GCAGGGTCACCATGACCAGGGACACGTCCATCAGCACAGCCTACATGGA
GCTGAGCAGGCTGAGATCTGACGACACGGCCGTGTATTACTGTGCGAGA
GAGGGGTCATCTACAGTAACCAGGGAGATGGACGTCTGGGGCCAAGGGA
CCACGGTCACCGTCTCTTCA
Seq ID No. 5: VH 6B2 amino acid sequence
QVQLVQSGAEVKKPGASVKVSCKASGYTFTGYYMHWVRQAPGQGLEWMG
WINPNSGGTNYAQKFQGRVTMTRDTSISTAYMELSRLRSDDTAVYYCAR
AYCGGDCPQDYYDYYGMDVWGQGTTVTVSS
Seq ID No. 6: VH 6B2 nucleic acid sequence
CAGGTGCAGCTGGTGCAGTCTGGGGCTGAGGTGAAGAAGCCTGGGGCCT
CAGTGAAGGTCTCCTGCAAGGCTTCTGGATACACCTTCACCGGCTACTA
TATGCACTGGGTGCGACAGGCCCCTGGACAAGGGCTTGAGTGGATGGGA
TGGATCAACCCTAACAGTGGTGGCACAAACTATGCACAGAAGTTTCAGG
GCAGGGTCACCATGACCAGGGACACGTCCATCAGCACAGCCTACATGGA
GCTGAGCAGGCTGAGATCTGACGACACGGCCGTGTATTACTGTGCGAGA
GCATATTGTGGTGGTGACTGCCCCCAGGATTACTATGACTACTACGGTA
TGGACGTCTGGGGCCAAGGGACCACGGTCACCGTCTCTTCA
Seq ID No. 7: VH 129D2 amino acid sequence
QVQLVQSGAEVKKPGASVKVSCKASGYTFTGYYMHWVRQAPGQGLEWMG
WINPNSGGTNYAQKFQGRVTMTRDTSISTAYMELSRLRSDDTAVYYCAS
RPLGYCGDGSCPFDYWGQGTLVTVSS
Seq ID No. 8: VH 129D2 nucleic acid sequence
CAGGTCCAGCTGGTGCAGTCTGGGGCTGAGGTGAAGAAGCCTGGGGCCT
CAGTGAAGGTCTCCTGCAAGGCTTCTGGATACACCTTCACCGGCTACTA
TATGCACTGGGTGCGACAGGCCCCTGGACAAGGGCTTGAGTGGATGGGA
TGGATCAACCCTAACAGTGGTGGCACAAACTATGCACAGAAGTTTCAGG
GCAGGGTCACCATGACCAGGGACACGTCCATCAGCACAGCCTACATGGA
GCTGAGCAGGCTGAGATCTGACGACACGGCCGTGTATTACTGTGCGAGC
CGACCCTTGGGGTATTGTGGTGATGGCAGCTGCCCCTTTGACTACTGGG
GCCAGGGAACCCTGGTCACTGTCTCTTCA

1. A human VH soluble scaffold derived from human germline gene VH1-02 capable of producing a VH domain expression library comprising at least 70% soluble clones.

2. A human VH scaffold according to claim 1 having at least 80%, 90%, 95% or 98% amino acid sequence identity with the sequence according to Seq ID No. 1.

3. A human VH scaffold or fragment thereof of claim 1 according to Seq ID No 1

4. A human VH scaffold according to claims 1-3 further comprising a CDR3 region.

5. A method for identifying a VH scaffold according to claims 1-3 comprising the steps of:
a) Obtaining a transgenic mouse capable of expressing heavy chain only antibodies (HcAbs) comprising human VH domains,
b) Obtaining a VH domain expression library from the mouse of step a) and expressing in E.coli,
c) Detecting soluble VH domains expressed in b) and
d) Determining the sequence of soluble VH domains to obtain a VH scaffold sequence.

6. The method of claim 5 wherein the VH domain library is expressed using ribosome display or phage display.

7. A method of constructing a VH domain expression library comprising the steps of;
a) Assembling the scaffolds according to claim 1-3 or 5 with a plurality of CDR3 nucleic acid sequences to obtain a VH domain repertoire
b) Expressing the VH domain repertoire to produce a VH domain library and selecting for functional VH domains against target antigen.

8. The method of claim 7 wherein the scaffold is defined according to Seq ID No. 1 or Seq ID No. 2.

9. The method of claim 8 wherein the scaffolds have at least 80%, 90%, 95% or 98% amino acid sequence identity with the sequences according to Seq ID No. 1 or Seq ID No. 2

10. The method of claims 7-9 wherein the selected VH domains are sequenced and/or expressed in a host cell.

11. The method of claims 7-10 comprising the step of CDR3 mutagenesis followed by further rounds of screening.

12. A human VH domain expression library comprising a scaffold according to claims 1-4.

13. A human VH domain expression library according to claim 12 comprising at least 10⁹ unique VH domains.

14. A human VH domain expression library according to claims 12-13 expressed on the surface of a filamentous bacteriophage.

15. An isolated human VH domain or fragment thereof comprising a scaffold as defined in claims 1-4.

16. A pharmaceutical composition comprising a human VH domain according to claim 15 in an effective amount for binding to a target antigen and a pharmaceutically acceptable excipient.
